(19) United States
(12) Patent Application Publication
(10) Pub. No.: US 2006/0084887 A1
(43) Pub. Date: Apr. 20, 2006

(54) Rapid, simple and humane method of bleeding mice

(76) Inventors: William T. Golde, Cutchouge, NY (US); Peter Gollobin, Matinecock, NY (US); Luis L. Rodriguez, Old Saybrook, CT (US)

Correspondence Address: USDA, ARS, OTT, 5601 SUNNYSIDE AVE, RM 4-1159, BELTSVILLE, MD, 20705-5131, US

(21) Appl. No.: 10/967,796

(22) Filed: Oct. 18, 2004

(51) Int. Cl. A61B 5/00; A61B 17/32

(52) U.S. Cl. 600/573; 606/181; 600/583

(57) ABSTRACT

A method and article for obtaining blood samples from a small animal wherein a disposable lancet device is used to optimize the humane collection of blood from the cheek-pouch of said small animal. The lancet device is a hand-held, generally flat lancet, having a handle portion at one end and a flat V-shaped point at its other end; the flat point has a length in the range of 4.0 to 6.0 mm.

### BACKGROUND OF THE INVENTION

1. Field of the Invention

This invention relates to an improved method and article for taking blood samples from small animals. More particularly, the invention relates to such a method utilizing a pre-sterilized, individually wrapped, disposable lancet having a flat point, the point having a length of 4.0 to 6.0 mm.

2. Description of the Relevant Art

Biomedical research using small animal models has contributed tremendously to the improvement of human and animal health. Small animals, most prominently rodents, provide an inexpensive platform for biological investigation as well as testing procedures and pharmaceuticals for safety and efficacy. Use of laboratory rodents has become more carefully regulated over the past several decades to insure that animal care and use in the research setting is as humane as possible. Researchers in all aspects of biology and medicine are governed by institutional animal care and use committees (IACUC) that insure the most humane use of these animals as possible.

One very important aspect of rodent models in research is the acquisition of blood samples for testing. Because of the small size of the animals, drawing blood samples is a challenge to the investigator. A number of methods are employed that pass the review of IACUC, but none are particularly humane or simple. For example, in the USA, the most common bleeding method is retro-orbital, puncturing the orbital sinus behind the eye. This method consistently yields a reasonable blood volume when the investigator is experienced and practiced. (2002. Laboratory Animal Medicine, 2^{nd} Edition, Fox et al., eds., Elsiever Science, New York, N.Y.). However, this method is banned in a number of countries because it is not considered humane.

A second common method is cardiac puncture. This procedure requires anesthesia, which may alter parameters of the experiment. Briefly, a small gauge needle is inserted into the ventricle and blood is slowly drawn out. This procedure requires an extremely practiced investigator and often animals do not survive the process. Again, this procedure is not simple and is only humane when the procedure goes very well with minimal damage along the needle track. Because the chance of losing animals is so prominent, investigators often use an inflated number of animals to accommodate loss during an experiment.

A third more simple method of bleeding mice is the tail clip. A piece of the tail is excised and blood harvested from the severed tail vein. This can be done repeatedly for a few sequential bleeds. The draw back is, that in order to leave enough tail for multiple future bleeds, only a small piece of tail is cut yielding a small blood sample of only a few drops (0.1 ml). This method is easily done without anesthesia. However, this procedure may not be considered humane, especially for multiple blood draws.

Finally, a method which is more humane and done without anesthesia is the saphenous vein puncture (Hem et al. 1998. Laboratory Animal 32: 364-368). This is a more complicated and time consuming method that involves immobilizing the animal with the rear legs accessible. The hair is shaved from the thigh using a small scalpel or razor. The saphenous vein of the thigh is evident and can be punctured with a 23 to 25 gauge needle. Blood is collected with a microvette capillary collection tube. These vessels have a maximum volume of 0.3 ml. A compress is held on the site to stop the bleeding.

This is a very humane method of blood collection; and since no anesthesia is required, there are no side effects to consider. However, this procedure requires extensive time working with each animal and is not compatible with large trials of pharmaceuticals or biologicals. The man hours required to do a large trial, 50 to 100 animals, would cause researchers to design smaller experiments using fewer animals. The investigators describing this method limit the amount of blood collected to 0.3 ml and in practice, usually less. Therefore, this would yield approximately 0.1 ml of serum and limit analysis to a few very small volume assays.

Thus, there is a need for an improved humane method of bleeding mice where utilization of the method can lessen the need for using large numbers of animals to compensate for animal losses and where utilizing the method allows for testing the animal as often as necessary, including sampling more than one time per day, and yields blood samples of sufficient volume for testing all necessary parameters.

### SUMMARY OF THE INVENTION

We have identified a method for humanely obtaining blood samples from small animals without causing damage to the animal, by utilizing a novel lancet which optimizes obtaining blood without injury to the animal.

In accordance with this discovery, it is an object of the invention to provide a method where blood samples could be drawn at frequent time intervals from small animals without causing damage to the animal.

It is also an object of the invention to provide a method where blood samples could be drawn from small animals in sufficient quantities for testing without causing damage to the animal.

Another object of the invention provides for an individually wrapped, disposable sterilized lancet which is easily manufactured and relatively inexpensive. Preferably, the lancet is a metal lancet, made from corrosion resistant stainless steel, and having dimensions which optimize the collection of blood from the cheek-pouch location of small animals humanely.

Other objects and advantages of this invention will become readily apparent from the ensuing description.

### BRIEF DESCRIPTION OF THE DRAWINGS

FIG. 1 is a perspective view of the disposable lancet 10 used in the present method.

FIG. 2 is a top elevational view of the disposable lancet 10 used in the present method.

FIG. 3 is a left side elevational view of the disposable lancet 10 used in the present method.

FIG. 4 is an elevational view of the longer side of the disposable lancet 10 used in the present method.

FIGS. 5 depicts the area at the rear end of the cheek pouch wherein a vascular bundle, a convenient and consistent source of blood, is located.

### DETAILED DESCRIPTION OF THE INVENTION

In contrast to methods described above, there is another method in use that is a more rapid and humane method for drawing blood samples from mice. has been in use by us and colleagues for a number of years. A small vascular bundle is located at the rear end of the cheek pouch of the laboratory mouse, Mus musculus, and can be a source of blood for sampling. The animal is held by the scruff of the neck with one cheek against the absorbent mat on the bench. Using a number 11 scalpel, the cheek facing the investigator is punched with enough force to poke a small stick hole and drops of blood exude from the stick point. MICROTAINER® tubes (B/D Medical Supplies, Franklin Lakes, N.J.) or some other equivalent, are used to collect the blood. Such tubes are provided with and without separating gel for serum collection and whole blood, in either heparin or EDTA as an anticoagulant. The position of the punch is critical to get a sufficient volume of blood in a very short time; and with practice, the investigator usually becomes proficient at positioning the scalpel. Volumes of up to 1.0 ml of blood can be collected from an adult mouse depending on the frequency of bleeding. Bleeding is stopped by applying a sterile gauze pad or tissue with compression for less than a minute. Soon after release, the mouse will groom and within a short time, no evidence of the blood draw remains. The scalpel can be cleaned with soap and rinsed with sterile water for reuse. However, with multiple uses, the scalpel can become dull; a new scalpel should be used after 3 or 4 mice.

A problem associated with this method, however, is that control of the scalpel during the stabbing process requires a fine touch, a technique that is perfected after many repetitions of the procedure. Investigators who are not yet proficient in the technique or those that only occasionally bleed mice are often frustrated by the inconsistencies of the procedure that they experience, i.e., sufficient amounts of blood are not recovered consistently, mice sometimes require intervention to arrest bleeding. Inconsistencies with regard to varying amounts of blood being recovered appear to be due to insufficient pressure being applied to the scalpel. In other situations, where a scalpel is too strongly applied, the cheek skin of the mouse can be cut, possibly requiring sutures and minor surgery to arrest the bleeding, or the inside of the cheek can be cut through causing profuse bleeding putting the animal in danger.

An alternative to using a scalpel to perform the procedure is to use a 19, 21, or 23 gauge needle, depending on the size of the mouse. The incidence of cheek lacerations is eliminated; however, the cut through to the inside of the cheek is still a risk. Further, a portion of the blood enters the barrel of the needle. Such blood can be harvested, but this requires a new needle for each animal. Cleaning and reuse of needles is not possible.

The method of bleeding mice by piercing the vascular bundle of the cheek pouch with a scalpel or needle has been used successfully by some experienced investigators; however, it is a difficult method to learn and consistent results in bleeding many mice may not occur. The procedure becomes time consuming because of necessary surgical intervention or because repeated attempts are required to obtain blood from mice from which no blood was collected. The process can be costly because a new scalpel or needle is required for each animal.

The method of the invention involves bleeding mice from the vascular bundle located in the mouse cheek pouch using the lancet device of the invention. The lancet device of the invention is based on the standard lancet used for obtaining small blood samples from humans; however, the length of the standard lancet, 3.0 mm, is too short for use in the mouse procedure. Therefore, 4.0 mm, 5.0 mm, and 5.5 mm lancets were produced and tested. The method is performed identically to the scalpel method described above with the advantage that the lancet's design controls the depth of the skin punch such that there is a high level of consistency. Few problems, other than the proper location of the punch, occurred. Blood volumes collected were always greater than 0.3 ml and bleeding was arrested once a sufficient volume was collected. The 4.0 mm lances worked very well on young mice, 2-6 weeks old; the 5 mm lances, on mice 2-6 months old; and 5.5 mm lances worked best on mice over 6 months old.

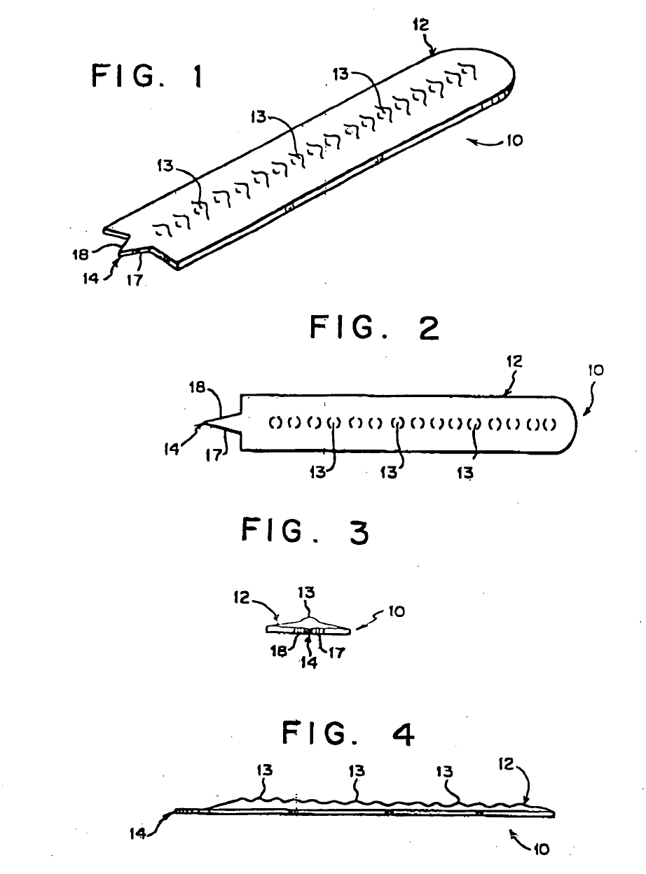

Thus, referring to FIGS. 1-4, an effective lancet according to the invention is a metal lancet, made from corrosion resistant stainless steel, its flat point having one or two sharpened edges. The lancet of the invention is a hand-held, disposable, elongated metal, generally flat lancet, having a handle portion 12 at one end and a flat V-shaped point 14 at its other end; said lancet has the following general dimensions: the flat point can range in length from 4.0 mm to 6.0 mm, depending on the size of the animal. The overall length can range from 20 mm to 60 mm; the width can range from 2.0 to 15.0 mm. The lancet is most desirably provided with a set of raised knurls 13 along the handle portion to aid in manipulating the device. The thickness of the lancet, excluding the raised knurls, is ideally in the range of 0.076 mm to 0.381 mm.

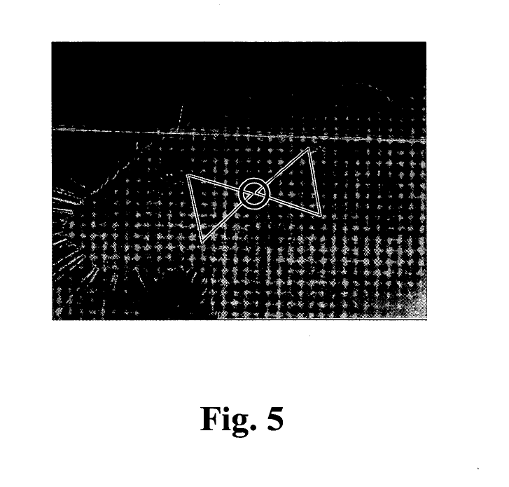

The mouse lancet of the invention eliminates the problems experienced when bleeding from the cheek pouch with either a scalpel or needle. The animal is held in the same manner as is used in the scalpel or needle procedure, and using the lancet of the invention, the cheek facing the investigator is punched with enough force to poke a small stick hole. When firm pressure is applied during the stick, sampling is very consistent for even occasional or inexperienced investigators performing the procedure. The position of the punch is critical to get a sufficient volume of blood in a very short time (see FIG. 5). The design of the lancet of the invention ensures that the animal does not experience a cut all the way through the inside of the cheek pouch and that a sufficient volume of blood is obtained humanely. This provides the researcher with the confidence to push hard enough so that too light a touch is no longer a problem. As with the needle or scalpel system, the lancets are individually wrapped and sterile to eliminate cross-contamination between animals. The lancets are relatively inexpensive; thus, they are more likely to be used on only a single animal.

Using this system, the same mouse can be bled daily as well as several times in a day. The volume of blood harvested when doing multiple bleedings in a day or daily bleeding should be adjusted to the total daily volumes recommended for animal safety and health for that size animal. The lancet device and method of the invention also allow for relatively rapid processing of many animals compared to the saphenous vein bleeding method where each animal must be shaved. An additional advantage is that there is no need for anesthesia, eliminating both effects on animal physiology and the need to tend to animals as the anesthetic wears off.

Compared to all mouse bleeding methods approved by IACUC guidelines, the mouse lancet for cheek pouch bleeding of the invention is by far the most humane, efficient, and economic method for bleeding laboratory mice. This style of lancet has been used for decades to draw blood from many mammalian species, especially humans, and results in very little pain and discomfort. Animal activist groups, concerned with the treatment of research animals, object to the current methods of drawing blood from small animals. Such groups should not have any objections to the use of the same type of lancets used on people for fingertip blood samples.

The utilization of this method and this particular lancet can improve scientific design and results in studies using laboratory mice and may be applied to other laboratory animals including rats, hamsters and gerbils.

All publications and patents mentioned in this specification are herein incorporated by reference to the same extent as if each individual publication or patent was specifically and individually indicated to be incorporated by reference.

The foregoing description and certain representative embodiments and details of the invention have been presented for purposes of illustration and description of the invention. It is not intended to be exhaustive or to limit the invention to the precise forms disclosed. It will be apparent to practitioners skilled in this art that modifications and variations may be made therein without departing from the scope of the invention.

1. An individually wrapped, disposable sterilized lancet wherein use of said sterilized lancet optimizes the humane collection of blood from the cheek-pouch location of small animals and wherein said lancet is a hand-held, disposable, elongated metal, generally flat lancet, having a handle portion at one end and a flat V-shaped point at its other end, said flat point of said lance having a length in the range of 4.0 to 6.0 mm, said lancet having an overall length in the range of 20-60 mm, said lancet having an overall width in the range of 2.0 to 15.0 mm, and said lancet most desirably provided with a set of raised knurls along the handle portion to aid in manipulating the device, the thickness of said lancet, excluding said raised knurls, being ideally in the range of 0.076 mm to 0.381 mm.

2-4. (canceled)

5. A hand-held, disposable, elongated, metal, generally flat lancet, having a handle portion at one end and a flat V-shaped point at its other end, said flat point of said lancet having a length in the range of 4.0 to 6.0 mm, said lancet having an overall length ranging from 20 mm to 60 mm, a width ranging from 2.0 to 15.0 mm, and a thickness ranging from 0.076 mm to 0.381 mm.

6. The lancet of claim 5 wherein said metal lancet is made from corrosion resistant stainless steel.

7. The lancet of claim 5 wherein said flat point has one or two sharpened edges.

8. The lancet of claim 5 wherein said lancet is provided with a set of raised knurls along the handle portion to aid in manipulating the device and wherein said lancet has a thickness in the range of 0.076 mm to 0.381 mm, excluding said raised knurls.

9. The lancet of claim 5 wherein said lancet is a sterilized lancet.

10. The lancet of claim 5 wherein said lancet is an individually wrapped, disposable, sterilized lancet.

11. The individually wrapped, disposable, sterilized lancet of claim 10, wherein use of said sterilized lancet optimizes the humane collection of blood from the cheek-pouch location, depicted in FIG. 5, of small animals.

12. A method for humanely obtaining one or more blood samples from a small animal by utilizing the lancet of any one of claims 5-10, wherein said lancet optimizes obtaining blood from the cheek-pouch location, depicted in FIG. 5, without injury to the animal, comprising:
(a) piercing the animal's cheek pouch, depicted in FIG. 5, with said lancet, and
(b) obtaining said blood sample.

13. The method of claim 12 wherein said blood sample or samples are drawn from the animal in sufficient quantities for testing without causing damage to the animal.

14. The method of claim 12 wherein said blood samples are drawn from the animal at frequent time intervals without causing damage to the animal.
